(19) United States
(12) Patent Application Publication
(10) Pub. No.: US 2024/0366112 A1
(43) Pub. Date: Nov. 7, 2024

(54) GAIT ANALYSIS METHOD, GAIT ANALYSIS DEVICE, AND COMPUTER-READABLE STORAGE MEDIUM

(71) Applicant: Wistron Corporation, New Taipei City (TW)

(72) Inventors: Ming Jie Li, New Taipei City (TW); Yi Yun Hsieh, New Taipei City (TW); Jia-Hong Zhang, New Taipei City (TW); Shih Chieh Lin, New Taipei City (TW); Shih-Yi Chao, New Taipei City (TW)

(73) Assignee: Wistron Corporation, New Taipei City (TW)

(21) Appl. No.: 18/336,028

(22) Filed: Jun. 16, 2023

(30) Foreign Application Priority Data
Apr. 25, 2023 (TW) 112115394

(51) Int. Cl. A61B 5/11; A61B 5/00

(52) U.S. Cl. CPC A61B 5/112; A61B 5/6829; A61B 5/7278; A61B 5/7289

(57) ABSTRACT

Disclosed are a gait analysis method, a gait analysis device, and a computer-readable storage medium. The method includes: obtaining consecutive N motion data; determining a plurality of probability distributions based on the N motion data, wherein the probability distributions respectively corresponds to a plurality of gait events; and determining an event time point of each gait event belonging to a specific step according to the plurality of probability distributions.

### CROSS-REFERENCE TO RELATED APPLICATION

This application claims the priority benefit of Taiwan application serial no. 112115394, filed on Apr. 25, 2023. The entirety of the above-mentioned patent application is hereby incorporated by reference herein and made a part of this specification.

### BACKGROUND

### Field of the Disclosure

The present disclosure relates to a gait evaluation technology, and in particular to a gait analysis method, a gait analysis device and a computer-readable storage medium.

### Description of Related Art

Gait analysis utilizes observation or scientific instruments to measure the condition of a subject under test while the subject under test is walking, and quantize the observed and measured results into parameters, and present various gait indices through objective data. Common gait indices include, for example, stride time, walking speed, and stride length.

In a variety of gait measurement methods, some studies adopted an optical imaging system, a force plate or a treadmill combined with a gait analysis system that are suitable for being arranged in an experimental environment. Such instruments, while being more accurate, are expensive and restricted to environmental constraints. Some other studies adopted a wearable device (such as a pressure-sensing insole or an inertial measurement unit (IMU)) worn on the foot of the subject under test. Such device not only is less expensive, but also is less restricted to the environment, and is more convenient in use.

In most studies, before calculating each gait index, it is necessary to use an algorithm to detect four gait events, and then computation is performed based on the relevant data of these gait events to obtain each gait index. The above-mentioned gait events are initial contact, foot-flat, heel-off and toe-off.

However, the current algorithms for detecting four gait events based on IMU and other wearable devices are mainly methodologies designed by researchers based on rule-based obtained from observing the raw data. This type of algorithms have the following limitations.

Limitation (1): It is required to set more identify rules and thresholds, and is often limited by ranges. For example, for a subject under test who walks fast with large strides, the detected acceleration value is relatively high; for a subject under test who walks slowly with small strides, the detected acceleration value is low. In other words, the acceleration values measured from different subjects under test will be scattered, so it is difficult to design a more appropriate rule.

Limitation (2): The tolerance to exceptions is small and the adaptability is low. For example, when detecting and collecting data about the gaits of two subjects under test, the threshold adopted for the subject under test with larger and more frequent strides is different from the threshold adopted for the other subject under test with smaller and less frequent strides. If only a set of common thresholds are adopted for computation, the detected gait events will be inaccurate, and even the events will be missed out when detection fails. In this case, a significant error will occur in the subsequent computation of the gait index, or even impossible to perform computation.

### SUMMARY OF THE DISCLOSURE

In view of the foregoing, the present disclosure provides a gait analysis method, a gait analysis device and a computer-readable storage medium, which are able to be used to solve the above technical problems.

Embodiments of the present disclosure provide a gait analysis method, which is suitable for a gait analysis device, including: obtaining consecutive N motion data, and N is a positive integer; determining a plurality of probability distributions based on the N motion data, and the plurality of probability distributions respectively correspond to a plurality of gait events; and determining event time points of various gait events belonging to a specific step according to the plurality of probability distributions.

An embodiment of the present disclosure provides a gait analysis device, which includes a storage circuit and a processor. The storage circuit stores a program code. The processor is coupled to the storage circuit to access the program code to: obtain consecutive N motion data, and N is a positive integer; determine a plurality of probability distributions based on the N motion data, and the plurality of probability distributions respectively correspond to a plurality of gait events; and determine event time points of various gait events belonging to a specific step according to the plurality of probability distributions.

Embodiments of the present disclosure provide a computer-readable storage medium, the computer-readable storage medium records an executable computer program, and the executable computer program is loaded by a gait analysis device to perform the following steps: obtaining consecutive N motion data, and N is a positive integer; determining a plurality of probability distributions based on the N motion data, and the plurality of probability distributions respectively correspond to a plurality of gait events; and determining event time points of various gait events belonging to a specific step according to the plurality of probability distributions.

### BRIEF DESCRIPTION OF THE DRAWINGS

FIG. 1 is a schematic diagram of a gait analysis device according to an embodiment of the present disclosure.

FIG. 2 is a flowchart of a gait analysis method according to an embodiment of the present disclosure.

FIG. 3 is a schematic diagram of motion data according to an embodiment of the present disclosure.

FIG. 4 is a flowchart of a training process according to an embodiment of the present disclosure.

FIG. 5 is a schematic diagram of a reference motion data string according to an embodiment of the present disclosure.

FIG. 6A is a schematic diagram of a preset probability distribution template according to an embodiment of the present disclosure.

FIG. 6B is a schematic diagram of a reference probability distribution for determining a gait event shown in FIG. 6A.

FIG. 7 is a flowchart of determining event time points of various gait events according to an embodiment of the present disclosure.

FIG. 8 is a schematic diagram of determining intersection over union variation diagram according to an embodiment of the present disclosure.

FIG. 9 is a flowchart of determining the event time points of various gait events based on the intersection over union variation diagram according to an embodiment of the present disclosure.

FIG. 10 is an application scenario diagram according to an embodiment of the present disclosure.

FIG. 11 is a schematic diagram illustrating deciding a window position according to an embodiment of the present disclosure.

FIG. 12 is a schematic diagram of a gait index dashboard according to an embodiment of the present disclosure.

### DESCRIPTION OF EMBODIMENTS

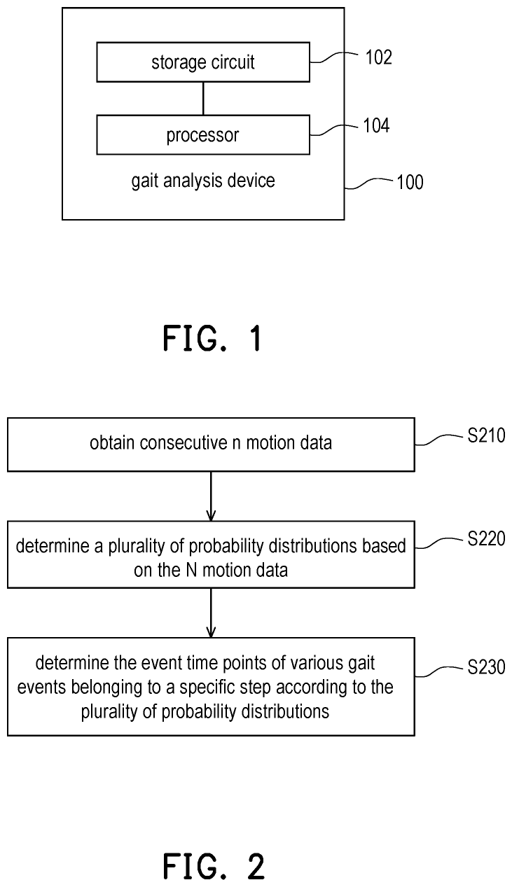

Please refer to FIG. 1, which is a schematic diagram of a gait analysis device according to an embodiment of the present disclosure. In different embodiments, the gait analysis device 100 may be implemented as various smart devices and/or computer devices, but is not limited thereto.

In FIG. 1, a gait analysis device 100 includes a storage circuit 102 and a processor 104.

The storage circuit 102 is, for example, any type of fixed or removable random access memory (RAM), a read-only memory (ROM), a flash memory, a hard disk or other similar device, or a combination of these devices, which may be adopted to record multiple program codes or modules.

The processor 104 is coupled to the storage circuit 102, and may be a general-purpose processor, a special-purpose processor, a conventional processor, a digital signal processor, a plurality of microprocessors, one or more microprocessors combined with digital signal processor core, a controller, a microcontroller, an application specific integrated circuit (ASIC), a field programmable gate array (FPGA) circuit, any other kind of integrated circuits, state machines, advanced RISC machine (ARM)-based processors, and the like.

In an embodiment of the present disclosure, the processor 104 is able to access the modules and program codes recorded in the storage circuit 102 to implement the gait analysis method provided by the present disclosure, the details of which are described below.

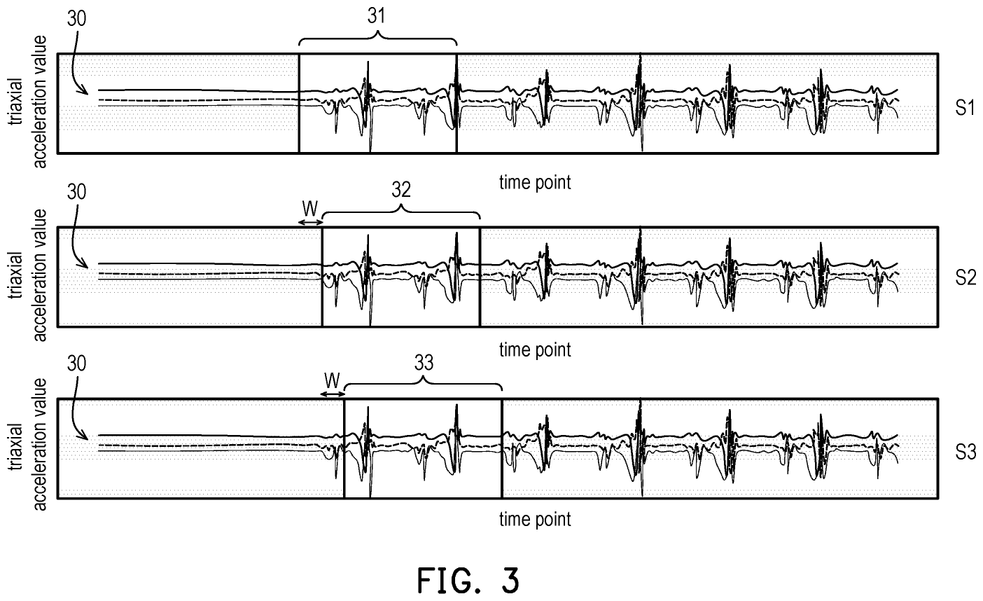

Please refer to FIG. 2, which is a flowchart of a gait analysis method according to an embodiment of the present disclosure. The method of this embodiment may be executed by the gait analysis device 100 in FIG. 1, and the details of various steps in FIG. 2 will be described below with the components shown in FIG. 1.

First, in step S210, the processor 104 obtains consecutive N motion data, and N can be any positive integer (e.g., 200) depending on the designer's requirements. In an embodiment of the present disclosure, the above-mentioned motion data is derived from a motion sensor (such as an IMU) set or worn on a certain foot (such as the instep or shoe of the subject under test), and each motion data includes 6 degrees of freedom (6DOF) values. In other words, the motion data may be used, for example, to characterize the motions of the feet of the subject under test during activities (e.g., walking).

In an embodiment of the present disclosure, the processor 104 may obtain corresponding consecutive N motion data at different stages/times to execute the method shown in FIG. 2.

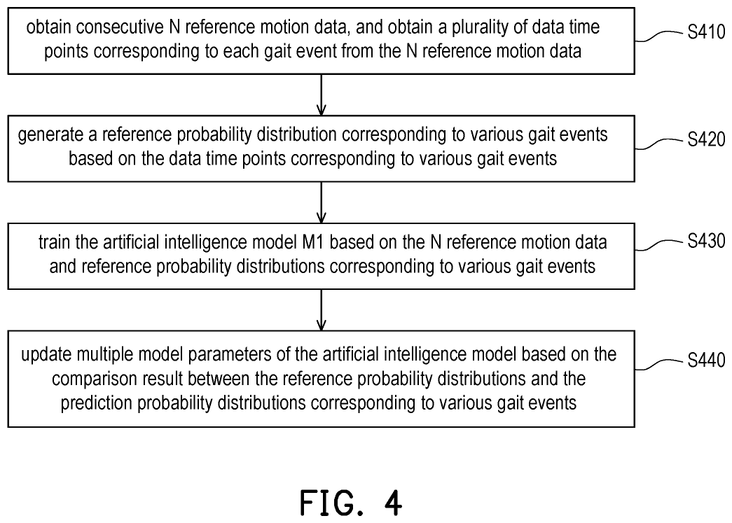

Please refer to FIG. 3, which is a schematic diagram of motion data according to an embodiment of the present disclosure. In FIG. 3, the motion data string 30 includes, for example, the motion data of 1000 time points (such as the triaxial acceleration value in the 6DOF value), and the motion data of the 1000 time points are, for example, obtained when the subject under test is walking, but not limited to. In this case, the processor 104 may, for example, acquire N motion data considered each time based on the concept of a sliding window.

For example, in the stage S1, the processor 104 may, for example, take corresponding N motion data from the motion data string 30 based on the window 31 (the length of which is N, for example) to execute the method in FIG. 2.

Afterwards, in the stage S2 next to the stage S1, the processor 104 may, for example, take corresponding N motion data from the motion data string 30 based on the window 32 (the length of which is N, for example) to execute the method in FIG. 2. The window 32 may be interpreted as an aspect of sliding the window 31 to the right by a distance W. Next, in the stage S3 next to the stage S2, the processor 104 may, for example, take corresponding N motion data from the motion data string 30 based on the window 33 (the length of which is N, for example) to execute the method in FIG. 2, and the window 33 may be interpreted as an aspect of sliding the window 32 to the right by the distance W.

In an embodiment of the present disclosure, the processor 104 may further obtain another motion data string (not shown) corresponding to the motion data string 30 from the above-mentioned motion sensor, and this other motion data string is, for example, (which is, for example, a triaxial angular velocity value in the 6DOF value). For this other motion data string, the processor 104 may further obtain a plurality of triaxial angular velocity values corresponding to stage S1 based on the teaching of FIG. 3, and then form N motion data corresponding to the stage S1 with the corresponding triaxial acceleration values.

For example, the window 31 includes the triaxial acceleration value corresponding to the 1000-th time point, and the processor 104 may combine the triaxial angular velocity value corresponding to the 1000-th time point with this triaxial acceleration value to become one of the N motion data corresponding to the window 31, but the disclosure is not limited thereto.

Next, in step S220, the processor 104 determines a plurality of probability distributions based on the N motion data, and the plurality of probability distributions respectively correspond to a plurality of gait events. In an embodiment, the plurality of gait events include, for example, initial contact, foot-flat, heel-off and toe-off, but not limited thereto.

In an embodiment, the processor 104 may feed the N motion data into an artificial intelligence model (hereinafter referred to as M1) that has undergone a training process, and the artificial intelligence model M1 generates the plurality of probability distributions in response to the N motion data.

In order to make the artificial intelligence model M1 have the above-mentioned function, the designer may train the artificial intelligence model M1 based on the following training process.

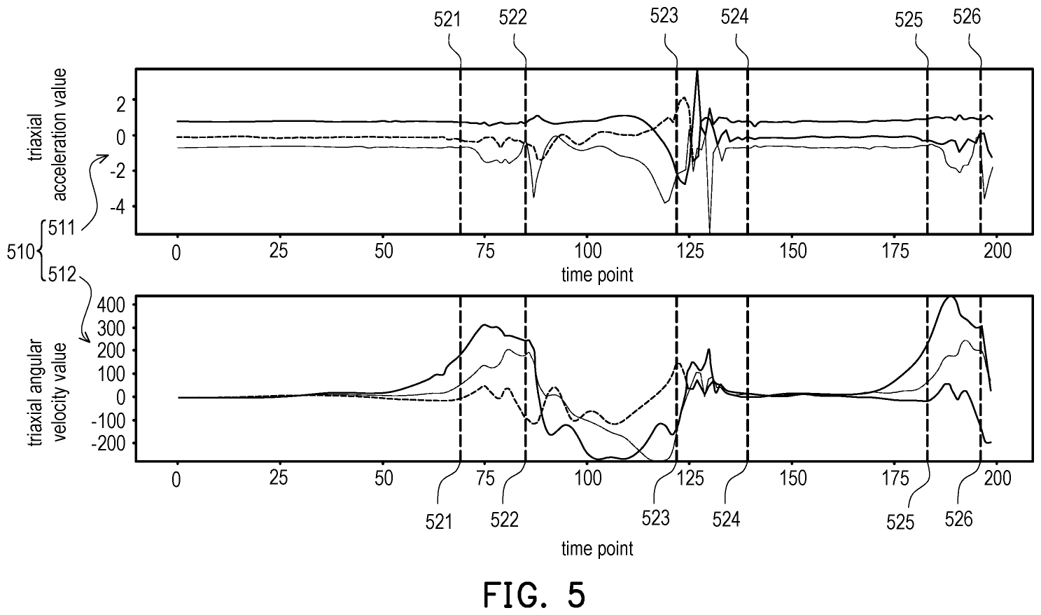

Please refer to FIG. 4, which is a flowchart of the training process according to an embodiment of the present disclosure. First, in step S410, the processor 104 may obtain consecutive N reference motion data, and obtain a plurality of data time points corresponding to each gait event from the N reference motion data.

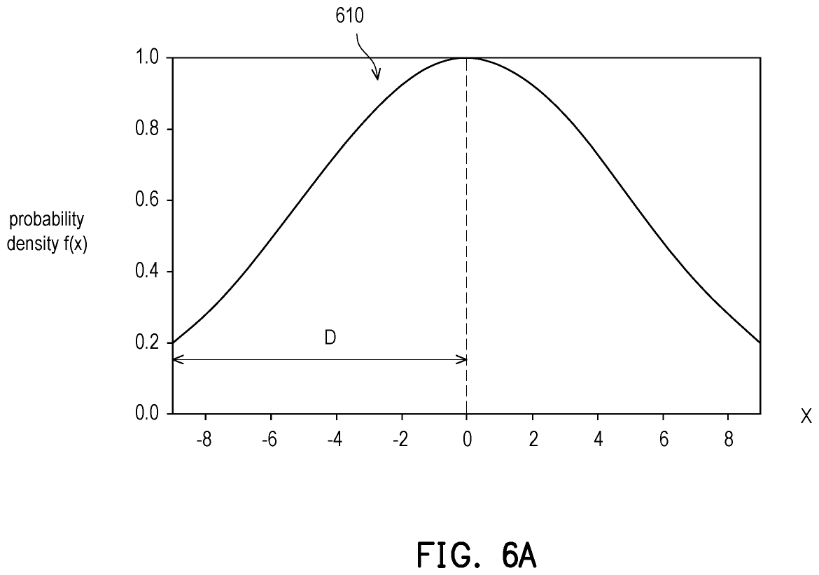

Please refer to FIG. 5, which is a schematic diagram of a reference motion data string according to an embodiment of the present disclosure. In FIG. 5, the processor 104 may obtain, for example, a reference motion data string 510 with a length of 200 time points, and the reference motion data string 510 may include a triaxial acceleration data string 511 and a triaxial angular velocity data string 512. In this embodiment, the reference motion data string 510 is, for example, measured by a motion sensor worn on the foot of a subject under test (hereinafter referred to as A), and the designer may utilize a photographic device to make an image/a video recording the walking action of the subject A under test, so as to know which gait event occurs to the foot of the subject A under test at which point in time by way of cross-comparison. Afterwards, the designer may mark the data time points corresponding to various gait events in the reference motion data string 510 accordingly.

For example, assuming that the designer determines that the gait events of heel-off and toe-off occurred to the foot of the subject A under test at time points 521 and 522 respectively based on the comparison of the videos recording the walking action of the subject A under test. Then the designer may mark time points 521 and 522 as data time points respectively corresponding to gait events of heel-off and toe-off.

In another example, assuming that the designer determines that the gait events of initial contact, foot-flat, heel-off and toe-off occurred to the foot of the subject A under test at time points 523 to 526 respectively based on the comparison of the videos recording the walking action of the subject A under test. Then the designer may mark time points 523 to 526 as data time points respectively corresponding to gait events of initial contact, foot-flat, heel-off and toe-off, but the disclosure is not limited thereto.

Afterwards, in step S420, the processor 104 may generate a reference probability distribution corresponding to various gait events based on the data time points corresponding to various gait events.

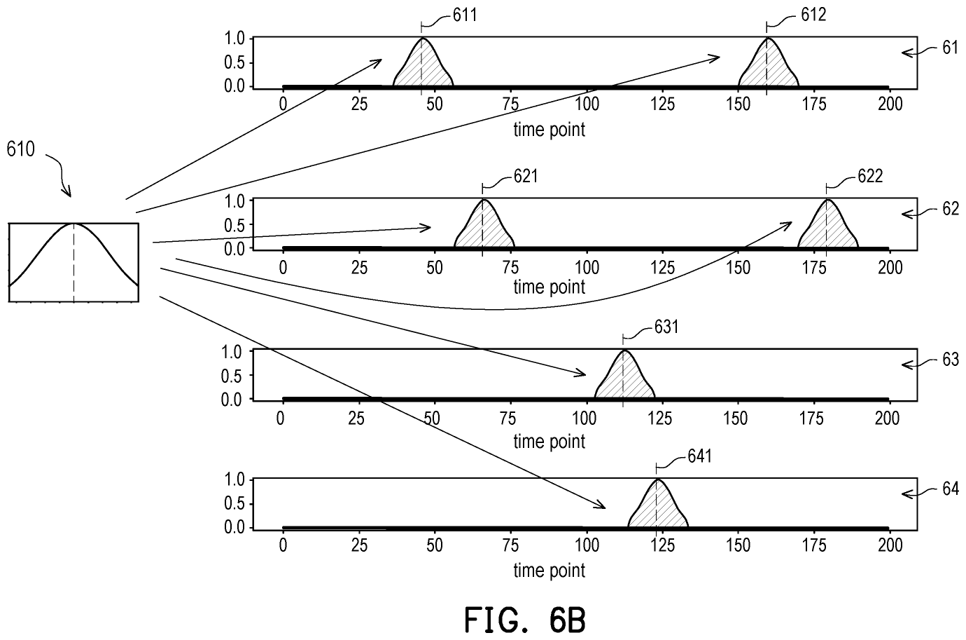

In an embodiment, the processor 104 may find at least one specific data time point corresponding to the j-th gait event (j is an index value) in the N reference motion data. Thereafter, the processor 104 may copy a preset probability distribution template to each specific data time point corresponding to the j-th gait event, so as to generate a reference probability distribution corresponding to the j-th gait event. For ease of understanding, further description is provided below with reference to FIG. 6A and FIG. 6B.

Please refer to FIG. 6A, which is a schematic diagram of a preset probability distribution template according to an embodiment of the present disclosure. In an embodiment, the preset probability distribution template 610 may be, for example, intercepted from a normal distribution with a specific standard deviation and a specific mean value. In FIG. 6A, the specific standard deviation and the specific mean value of the considered normal distribution are, for example, 5 and 0, respectively, but not limited thereto. Based on the above, the processor 104 may take D data points located on the left of the specified mean value (D may be determined depending on the designer's needs), D data points located on the right of the specified mean value, and data points corresponding to the specified mean value to compose the preset probability distribution template 610 (which, for example, includes 2D+1 data points), but the disclosure is not limited thereto.

Please refer to FIG. 6B, which is a schematic diagram of the reference probability distribution for determining the gait events shown in FIG. 6A. In the scenario of FIG. 6B, assuming that in a considered reference motion data string (not shown, hereinafter referred to as B), the time points 611 and 612 are marked as data time points corresponding to the gait event of initial contact. Then, after first establishing a reference probability distribution 61 corresponding to a gait event of initial contact (the length of which corresponds to the considered reference motion data string B), the processor 104 may copy the preset probability distribution template 610 to the time points 611 and 612 in the reference probability distribution 61. In this way, a reference probability distribution 61 corresponding to the gait event of initial contact may be formed.

In addition, assuming that in the considered reference motion data string B, the time points 621 and 622 are marked as data time points corresponding to the gait event of foot-flat, after first establishing a reference probability distribution 62 corresponding to a gait event of foot-flat (the length of which corresponds to the considered reference motion data string B), the processor 104 may copy the preset probability distribution template 610 to the time points 621 and 622 in the reference probability distribution 62. In this way, a reference probability distribution 62 corresponding to the gait event of foot-flat may be formed.

Additionally, assuming that in the considered reference motion data string B, the time point 631 is marked as a data time point corresponding to the gait event of heel-off, after first establishing a reference probability distribution 63 corresponding to a gait event of heel-off (the length of which corresponds to the considered reference motion data string B), the processor 104 may copy the preset probability distribution template 610 to the time point 631 in the reference probability distribution 63. In this way, a reference probability distribution 63 corresponding to the gait event of heel-off may be formed.

Similarly, assuming that in the considered reference motion data string B, the time point 641 is marked as a data time point corresponding to the gait event of toe-off, after first establishing a reference probability distribution 64 corresponding to a gait event of toe-off (the length of which corresponds to the considered reference motion data string B), the processor 104 may copy the preset probability distribution template 610 to the time point 641 in the reference probability distribution 64. In this way, a reference probability distribution 64 corresponding to the gait event of toe-off may be formed.

Please refer to FIG. 4 again, after generating the reference probability distributions corresponding to various gait events (such as the reference probability distributions 61 to 64 in FIG. 6B), in step S430, the processor 104 may train the artificial intelligence model M1 based on the N reference motion data and reference probability distributions corresponding to various gait events, and the artificial intelligence model M1 may generate prediction probability distributions corresponding to various gait events in response to the N reference motion data.

In an embodiment of the present disclosure, assuming that the considered gait events include the previously mentioned four states, namely, initial contact, foot-flat, heel-off and toe-off, the artificial intelligence model M1 may generate four corresponding prediction probability distributions.

In step S440, the processor 104 may update multiple model parameters of the artificial intelligence model M1 based on the comparison results between the reference probability distributions and the prediction probability distributions corresponding to various gait events. The plurality of model parameters may be determined according to the selected artificial intelligence model M1. For example, if a certain type of neural network is selected to implement the artificial intelligence model M1, the multiple model parameters are, for example, the weights of each neuron in this type of neural network, but the disclosure is not limited thereto.

In an embodiment, if the training of the artificial intelligence model M1 is nearly completed, the four prediction probability distributions generated through the training should be similar to the reference probability distributions 61 to 64 in FIG. 6B respectively. In contrast, if the training of the artificial intelligence model M1 has not been completed, the four prediction probability distributions generated through the training should be very different from the reference probability distributions 61 to 64 in FIG. 6B. Based on these differences (comparison results), the artificial intelligence model M1 will be trained continuously.

Based on the above, step S440 may be interpreted as updating multiple model parameters of the artificial intelligence model M1 based on the difference between the above-mentioned prediction probability distribution and the corresponding reference probability distribution. In an embodiment, the difference may be characterized as an L2 loss between the prediction probability distribution and the corresponding reference probability distribution, and the concept/method of estimating the L2 loss and updating model parameters accordingly may be derived from related art, no further details will be incorporated herein.

Based on the above, when the processor 104 feeds the N motion data into the trained artificial intelligence model M1 in step S220 of FIG. 2, the artificial intelligence model M1 correspondingly generates probability distributions corresponding to the number of considered gait events respectively, and the aspects of these probability distributions may be similar to the reference probability distributions 61 to 64 in FIG. 6B.

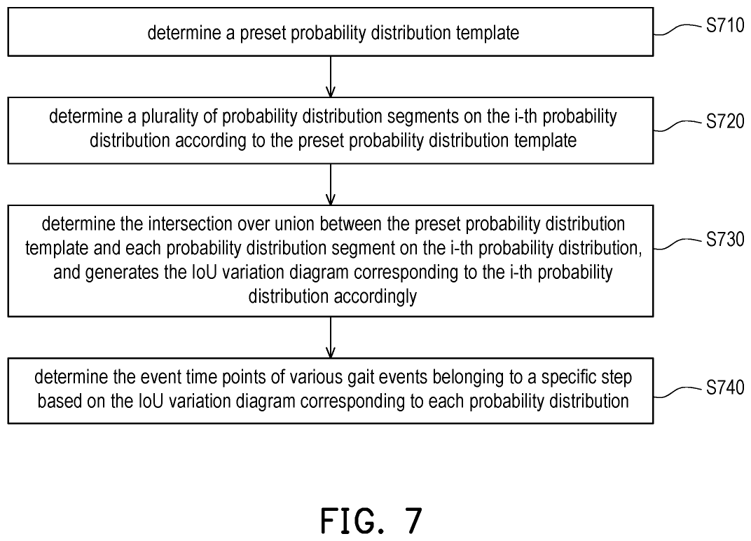

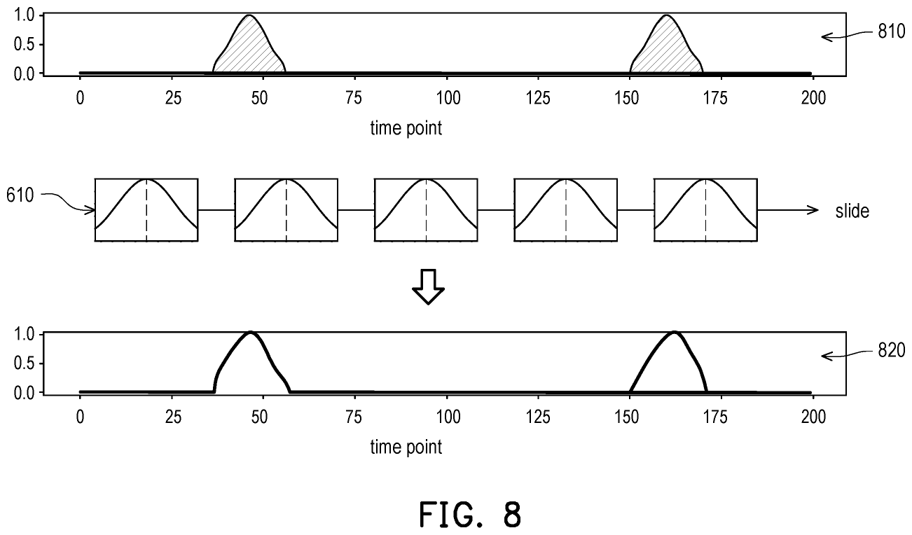

Afterwards, in step S230, the processor 104 determines the event time points of various gait events belonging to a specific step according to the plurality of probability distributions, and the relevant details of step S230 will be described with reference to FIG. 7 and FIG. 8.

Please refer to FIG. 7 and FIG. 8, FIG. 7 is a flowchart of determining the event time points of various gait events according to an embodiment of the present disclosure, and FIG. 8 is a schematic diagram of determining intersection over union variation diagram in an embodiment of the present disclosure.

First, in step S710, the processor 104 may determine a preset probability distribution template, and the preset probability distribution template is, for example, the preset probability distribution template 610 adopted in the training process. For ease of description, it is assumed that the preset probability distribution template mentioned in FIG. 7 is the preset probability distribution template 610 shown in FIG. 6, but the disclosure is not limited thereto.

Next, in step S720, the processor 104 determines a plurality of probability distribution segments on the i-th probability distribution (i is an index value) according to the preset probability distribution template 610.

In FIG. 8, assuming that the i-th probability distribution considered is a probability distribution 810, the processor 104 may determine multiple sliding positions of the preset probability distribution template 610 on the probability distribution 810 based on the concept of a sliding window. For example, the preset probability distribution template 610 is slid according to time points, and the sliding positions corresponding to various time points are different, and the segments of the probability distribution 810 corresponding to various sliding positions of the preset probability distribution template 610 are determined as the multiple probability distribution segments, but the disclosure is not limited thereto.

Afterwards, in step S730, the processor 104 determines the intersection over union (IoU) between the preset probability distribution template 610 and each probability distribution segment on the i-th probability distribution, and generates the IoU variation diagram 820 corresponding to the i-th probability distribution accordingly.

In an embodiment, each time the processor 104 slides the preset probability distribution template 610 to a sliding position, the IoU between the preset probability distribution template 610 and the corresponding probability distribution segment in the probability distribution 810 may be estimated. Based on the above, the IoU variation diagram 820 may be interpreted as a combination of the IoU corresponding to various sliding positions, but the disclosure is not limited thereto.

For each probability distribution obtained in step S220, the processor 104 is able to generate a corresponding IoU variation diagram according to the above teaching. In the case that the considered gait events include the previously mentioned four states, namely, initial contact, foot-flat, heel-off and toe-off, the processor 104 may generate four IoU variation diagrams corresponding to the four states.

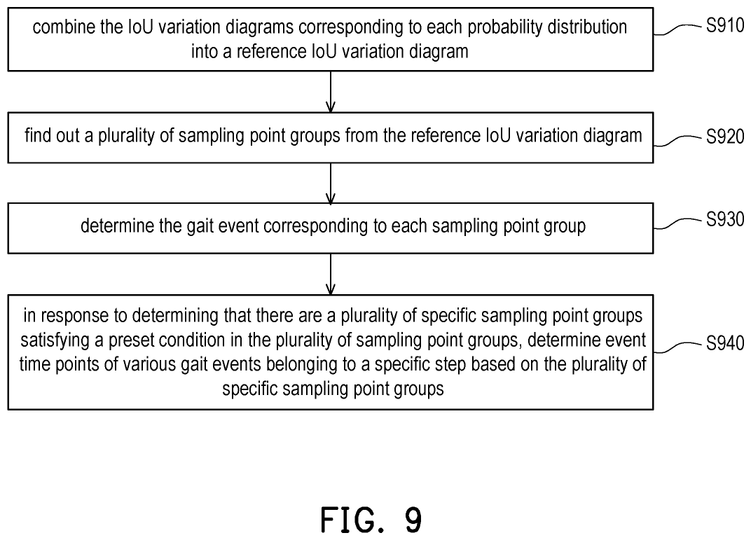

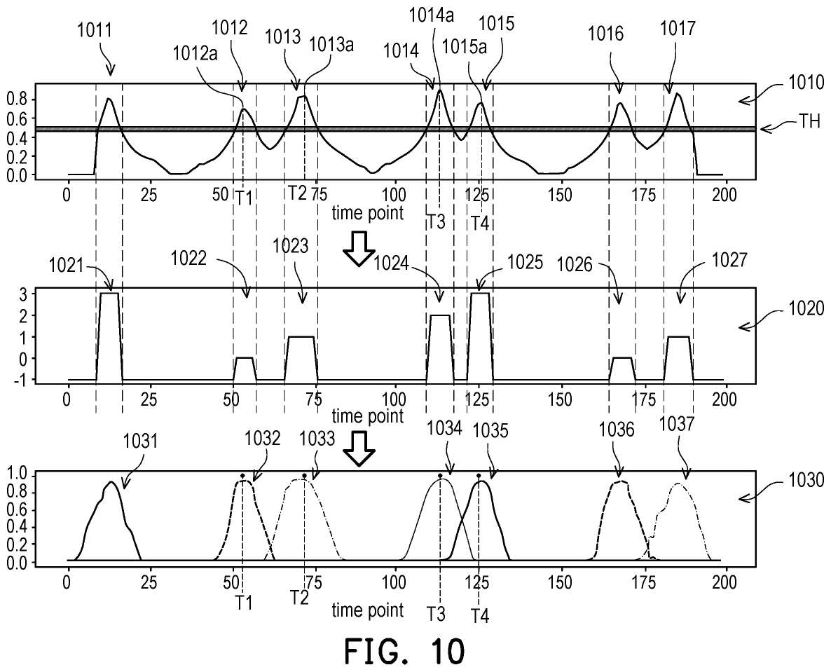

Afterwards, in step S740, the processor 104 determines the event time points of various gait events belonging to a specific step based on the IoU variation diagram corresponding to each probability distribution, the relevant details of step S740 will be described with reference to FIG. 9 and FIG. 10.

Referring to FIG. 9 and FIG. 10, FIG. 9 is a flowchart of determining the event time points of various gait events based on the IoU variation diagram according to an embodiment of the present disclosure, and FIG. 10 is an application scenario diagram according to an embodiment of the present disclosure.

In step S910, the processor 104 may combine the IoU variation diagrams corresponding to each probability distribution into a reference IoU variation diagram, and the reference IoU variation diagram includes a plurality of IoU sampling points. For example, after the processor 104 is able to generate four IoU variation diagrams respectively corresponding to the above four aspects, the processor 104 may combine the four IoU variation diagrams point by point into a corresponding reference IoU variation diagram, such as the reference IoU variation diagram 1010 shown in FIG. 10, and the reference IoU variation diagram 1010 includes, for example, 200 IoU sampling points, but the disclosure is not limited thereto.

In step S920, the processor 104 may find out a plurality of sampling point groups from the reference IoU variation diagram, and the IoU corresponding to the IoU sampling point in each sampling point group is higher than the preset threshold.

In the scenario of FIG. 10, the preset threshold value TH is 0.5, and the processor 104 may find out the sampling point groups 1011 to 1017 from the reference IoU variation diagram 1010, and each sampling point group 1011 to 1017 includes a plurality of consecutive IoU sampling points, and the IoUs corresponding to the IoU sampling points in each sampling point group 1011 to 1017 are all higher than the preset threshold TH.

In step S930, the processor 104 may determine the gait event corresponding to each sampling point group. In the scenario of FIG. 10, since the reference IoU variation diagram 1010 is formed by combining the IoU variation diagrams corresponding to different gait events, the processor 104 may determine the IoU variation diagrams of specific gait events that mainly correspond to the sampling point groups 1011 to 1017.

In an embodiment of the present disclosure, assuming that the four gait events such as initial contact, foot-flat, heel-off and toe-off are represented by 0 to 3 respectively, the determining result of step S930, for example, may be shown as a gait event correspondence diagram 1020.

For example, assuming that the sampling point group 1011 mainly corresponds to toe-off, the processor 104 may characterize the sampling point group 1011 as 3 corresponding to the toe-off, and correspondingly set the segment 1021 corresponding to the sample point group 1011 in the gait event correspondence diagram 1020.

In another example, assuming that the sampling point groups 1012 to 1015 mainly correspond to initial contact, foot-flat, heel-off and toe-off respectively, the processor 104 may characterize the sampling point groups 1012 to 1015 as 0 to 3 corresponding to the initial contact, foot-flat, heel-off and toe-off respectively, and correspondingly set the segments 1022 to 1025 corresponding to the sample point groups 1012 to 1015 in the gait event correspondence diagram 1020.

Furthermore, assuming that the sampling point groups 1016 and 1017 mainly correspond to initial contact and foot-flat respectively, the processor 104 may characterize the sampling point groups 1016 and 1017 as 0 and 1 corresponding to initial contact and foot-flat respectively, and correspondingly set the segments 1026 and 1027 corresponding to the sample point groups 1016 and 1017 in the gait event correspondence diagram 1020.

In an embodiment, the processor 104 may determine whether there are a plurality of specific sampling point groups satisfying a preset condition among the plurality of sampling point groups 1011 to 1017.

In an embodiment, it is assumed that the considered gait events include the 1-st gait event to the K-th gait event (K is a positive integer).

In this case, in response to determining that the consecutive K sampling point groups in the sampling point groups 1011 to 1017 correspond to the 1-st gait event to the K-th gait event in sequence, the processor 104 may determine that the consecutive K sampling point groups are specific sampling point groups satisfying the preset condition.

In the previous example, K is, for example, 4, and the 1-st gait event to the K-th gait event may sequentially include initial contact, foot-flat, heel-off and toe-off, but the disclosure is not limited thereto.

In the scenario of FIG. 10, since the sampling point groups 1012 to 1015 correspond to initial contact, foot-flat, heel-off and toe-off in sequence (that is, the 1-st gait event to the K-th gait event), the processor 104 may consider the sampling point groups 1012 to 1015 as specific sampling point groups satisfying the preset condition.

Specifically, a complete step should in principle include initial contact, foot-flat, heel-off and toe-off in sequence. Therefore, when a specific sampling point group meeting the preset condition appears in the reference IoU variation diagram 1010, it means that there is data corresponding to a complete step in the reference IoU variation diagram 1010.

In an embodiment of the present disclosure, the steps corresponding to the sampling point groups 1012 to 1015 are referred to as specific steps below, but the disclosure is not limited thereto.

In other embodiments, if the reference IoU variation diagram 1010 only includes sampling point groups 1013 to 1017, the processor 104 may determine that there is no specific sampling point group satisfying the preset condition, but the disclosure is not limited thereto.

Based on the above, in step S940, in response to determining that there are a plurality of specific sampling point groups satisfying a preset condition in the plurality of sampling point groups (for example, sampling point groups 1012 to 1015 corresponding to initial contact, foot-flat, heel-off and toe-off in sequence), the processor 104 may determine event time points of various gait events belonging to a specific step based on the plurality of specific sampling point groups.

In an embodiment, the processor 104 may determine that the above-mentioned specific sampling point group (for example, sampling point groups 1012 to 1015) belongs to a specific step, and find the specific sampling point in the j-th specific sampling point group, the j-th specific sampling point group corresponds to the j-th gait event.

In the scenario of FIG. 10, the sampling point groups 1012 to 1015 are respectively, for example, the first specific sampling point group to the fourth specific sampling point group, and the processor 104 may find the corresponding specific sampling point from each sampling point group 1012 to 1015.

In an embodiment, the specific sampling point of each sampling point group 1012 to 1015 is, for example, the IoU sampling point with the highest IoU in the corresponding sampling point group, but the disclosure is not limited thereto. For example, the processor 104 may find a specific sampling point 1012a from the sampling point group 1012, which is, for example, the IoU sampling point with the highest IoU in the sampling point group 1012. In another example, the processor 104 may find a specific sampling point 1013a from the sampling point group 1013, which is, for example, the IoU sampling point with the highest IoU in the sampling point group 1013. Based on the principle of similarity, the processor 104 may find specific sampling points 1014a and 1015a respectively from the sampling point groups 1014 and 1015, but the disclosure is not limited thereto.

Afterwards, the processor 104 takes the time point corresponding to the specific sampling point of the j-th specific sampling point group as the event time point of the j-th gait event belonging to a specific step. For example, the processor 104 takes the time point T1 of the specific sampling point 1012a as the event time point of initial contact of a specific step, takes the time point T2 of the specific sampling point 1013a as the event time point of foot-flat of the specific step, takes the time point T3 of the specific sampling point 1014a as the event time point of heel-off of the specific step, and takes the time point T4 of the specific sampling point 1015a as the event time point of toe-off of the specific step.

In FIG. 10, the probability distribution of each gait event may be integrated into a probability distribution integrated diagram 1030, in which the probability distribution components 1031 and 1035 may correspond to the probability distribution of toe-off, the probability distribution components 1032 and 1036 may correspond to the probability distribution of initial contact, the probability distribution components 1033 and 1037 may correspond to the probability distribution of the foot-flat, and the probability distribution component 1034 may correspond to the probability distribution of heel-off, but the disclosure is not limited thereto.

It may be seen from the probability distribution integrated diagram 1030 that the probability distribution components 1032 to 1035 correspond to the sampling point groups 1012 to 1015 respectively, and the time points T1 to T4 respectively correspond to data points having the highest value among the probability distribution components 1032 to 1035.

As can be seen from the above, different from the conventional method of determining gait events with the rule-based concept, the method provided in the embodiment of the present disclosure is able to find the gait events belonging to the same step and the corresponding event time points based on the relevant probability distributions after collecting motion data of the subject under test when the subject under test is walking. Based on the above, the method of the embodiment of the present disclosure may not only be adapted to the subjects under test who walk in different ways, but also may measure the gait events more accurately.

In an embodiment, the processor 104 may obtain N motion data corresponding to different stages according to the sliding window method shown in FIG. 3. However, in other embodiments, the processor 104 may also determine the window position corresponding to the next stage according to whether the event time point of the gait event belonging to the same step is successfully found out based on the N motion data corresponding to the certain stage.

In an embodiment, in response to determining that the event time point of each gait event corresponding to a specific step has been found based on the N motion data, the processor 104 may obtain the event time point of the K-th gait event corresponding to the specific step. Thereafter, the processor 104 may obtain other consecutive N motion data (that is, the motion data used in the next stage) from the event time point of the K-th gait event corresponding to the specific step, and determine a plurality of other probability distributions based on the N other motion data, the plurality of other probability distributions respectively correspond to the plurality of gait events. Then, the processor 104 may determine the event time points of various gait events belonging to other specific steps according to the plurality of other probability distributions.

In another embodiment, in response to determining that the event time point of each gait event corresponding to a specific step cannot be found based on the N motion data, the processor 104 obtains other consecutive N motion data (that is, the motion data used in the next stage), and the first other motion data among the N other motion data has a time difference with the first motion data among the N motion data. Thereafter, the processor 104 may determine a plurality of other probability distributions based on the N other motion data, and determine event time points of various gait events belonging to other specific steps according to the plurality of other probability distributions.

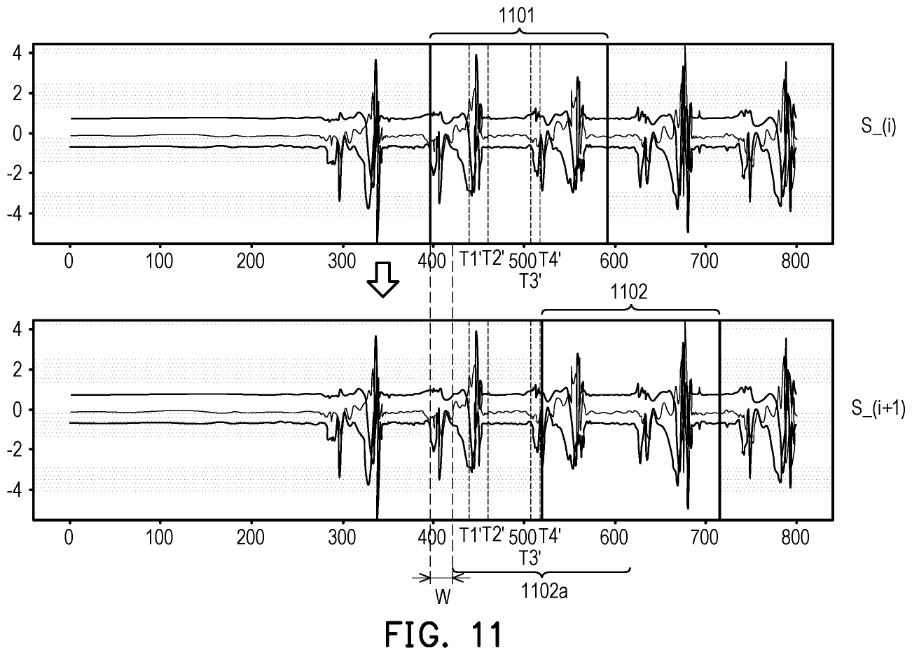

In order to make the above concept easier to understand, further description is provided below with reference to FIG. 11. FIG. 11 is a schematic diagram illustrating deciding a window position according to an embodiment of the present disclosure. In FIG. 11, it is assumed that the processor 104 obtains the corresponding N motion data through the window 1101 in stage S_(i), and successfully determines time points T1′ to T4′ of initial contact, foot-flat, heel-off and toe-off belonging to the same step. Based on the above, in the next stage S_(i+1), the processor 104 may obtain the N motion data used in the stage S_(i+1) at the time point T4′ of toe-off to execute the method in FIG. 2.

In short, if the time points T1′ to T4′ corresponding to the same step are successfully determined based on the N motion data corresponding to the window 1101 in the stage S_(i), then the processor 104 may obtain the corresponding N motion data in the next stage S_(i+1) directly based on the window 1102 (the length of which is the same as that of the window 1101), and the window 1102 starts at the time point T4′ corresponding to toe-off (that is, the event time point of the K-th gait event).

In other words, the processor 104 does not slide the window with a fixed distance W as shown in FIG. 3, but takes the time point T4′ corresponding to toe-off as the starting point of the next window.

Furthermore, if the window 1101 is only slid by a fixed distance W to determine the window 1102a of the next stage, since the windows 1101 and 1102a both involve the motion data corresponding to the time points T1′ to T4′, the step may be determined repeatedly. However, in FIG. 11, since the window 1102 does not involve the motion data corresponding to the time points T1′ to T4′, it is possible to avoid repeated determining of step. Moreover, since the mechanism shown in FIG. 11 makes it possible to avoid analyzing the same motion data, gait events may be determined with better efficiency.

In another embodiment, if the event time point of each gait event corresponding to the same step is not successfully determined based on the N motion data corresponding to the window 1101 in the stage S_(i), the processor 104 may obtain the N motion data used in the stage S_(i+1) based on the window 1102a. In this case, there will be a fixed time difference (corresponding to the distance W) between the first motion data obtained based on the window 1101 and the first motion data obtained based on the window 1102a, but the disclosure is not limited thereto.

In an embodiment, after determining the event time points (such as time points T1 to T4 in FIG. 10) of each gait event belonging to a specific step, the processor 104 may further determine one or more gait indices based on the event time points of various gait events belonging to a specific step.

Generally speaking, a complete gait cycle includes a stance phase, a swing phase and a double stance phase, in which the stance phase is a time interval when the feet touch the ground (which at least includes the four gait events mentioned earlier), the swing phase is a time interval when the foot leaves the ground, and the double stance phase is a time interval when both feet touch the ground simultaneously.

In different embodiments, the gait index includes, for example, a cycle time, a stance time, a swing time, a stance duration, a swing duration, a cadence, a single support time, a stride length, and a walking speed. Descriptions for the above gait indices may be derived from the content in Table 1 below.

TABLE 1

| Gait Indices and | |
|---|---|
| Units | Descriptions of gait indices |
| cycle time (sec.) | The sum of the stance time and swing time, also |
| | known as stride time |
| stance time (sec.) | Time spent standing on one leg |
| swing time (sec.) | Time spent swinging one leg in the air |
| stance duration (%) | Percentage of time spent standing on one leg in the |
| | cycle time |
| swing duration (%) | Percentage of time spent swinging one leg in the |
| | cycle time |
| cadence (step/min.) | Number of steps in one minute |
| single support time | Time of standing with support of one leg on the |
| (sec.) | ground |
| stride length (cm) | Length of stride |
| walking speed | Speed of walking |
| (km/h) | |

In an embodiment of the present disclosure, since the provided method may be able to estimate the event time point of each gait event more accurately, it is possible to make the gait index that is subsequently estimated more accurate accordingly.

For the description of the method/formula of estimating the required gait index based on the event time point of each gait event, reference may be made to documents of related art.

In an embodiment, it is assumed that the event time points corresponding to initial contact, foot-flat, heel-off and toe-off corresponding to a specific step i are ICᵢ, FFᵢ, HOᵢ, and TOᵢ, and each gait index in Table 1 may be calculated according to the content in Table 2 below, for example.

TABLE 2

| Gait Indices and | |
|---|---|
| Units | Formula |
| cycle time (sec.) | T_{cycle,i} = T_{stance,i} + T_{swing,i} |
| stance time (sec.) | T_{stance,i} = TOᵢ − ICᵢ |
| swing time (sec.) | T_{swing,i} = ICᵢ₊₁ − TOᵢ |
| stance duration (%) | $\frac{{T}_{stance , i}}{{T}_{cycle , i}} * 100 \%$ |
| swing duration (%) | $\frac{{T}_{swing , i}}{{T}_{cycle , i}} * 100 \%$ |
| cadence (step/min.) | $\frac{120}{{T}_{cycle , i}}$ |
| single support time | HOᵢ − FFᵢ |
| (sec.) | |
| stride length (cm) | Lᵢ = \sqrt{P_{c,i,x}(t_{rest,i+1})² + P_{c,i,y}(t_{rest,i+1})²} |
| walking speed | v(t) = ∫ₜ₌₀^{t} Acc_{extract} dt (Acc_{extract} is acceleration) |
| (km/h) | |

In Table 2, p_{c,i}(t_{rest,i+1}) is characterized as:

p_{c,i}(t_{rest,i+1})=∫ₜ₌ₜ_{_{rest,i+1}}^{t}^{_{rest,i+1}}v_{c,i}(t) dt, in which

$$\\ {v}_{c , i} ( {t}_{k} ) = {v}_{i} ( {t}_{k} ) - \frac{{t}_{k} - {t}_{rest , i}}{{t}_{rest , i + 1} - {t}_{rest , i}} {v}_{i} ( {t}_{rest , i + 1} ) ;$$

tₖ∈[t_{rest,i}, t_{rest,i+1}], and restᵢ=(HOᵢ−FFᵢ)/2.

In the previous embodiments, since the motion sensor is assumed to be disposed on a foot of the subject under test, the motion data considered in the above embodiments all correspond to the motions of a single foot. In this case, the obtained gait indices correspond to a single foot, for example.

In an embodiment, the above-mentioned specific step corresponds to the first foot of the subject under test, for example, and the processor 104 may determine at least one first gait index corresponding to the first foot according to the event time point of each gait event belonging to the specific step. In addition, for the second foot of the subject under test, the processor 104 may obtain at least one corresponding second gait index according to the content described in the previous embodiment.

Thereafter, the processor 104 may determine the gait asymmetry between the first foot and the second foot based on the first gait index and the second gait index.

In an embodiment, the feet of the subject under test may be respectively provided with a motion sensor, which may be respectively used to collect the motion data of the feet of the subject under test. In this case, the processor 104 may correspondingly determine the individual gait indices of the feet of the subject under test, and determine the symmetry of the feet of the subject under test accordingly when the subject under test is walking. Generally speaking, if the values of the feet of the subject under test are closer to each other in the same gait index, it means that the symmetry of the feet of the subject under test in this gait index is higher (i.e., the asymmetry is lower).

For example, the closer the walking speed of the feet of the subject under test is, the more symmetrical the walking speed of the feet of the subject under test is. In another example, the closer the cadence of the feet of the subject under test is, the more symmetrical the cadence of the feet of the subject under test is.

In an embodiment, the gait analysis device 100 may further include a display device (such as various screens and/or a human-machine interface), and the processor 104 may control the display device to display the above-mentioned gait asymmetry. In an embodiment, the processor 104 may further present various gait indices of both feet on the display device in the form of a dashboard.

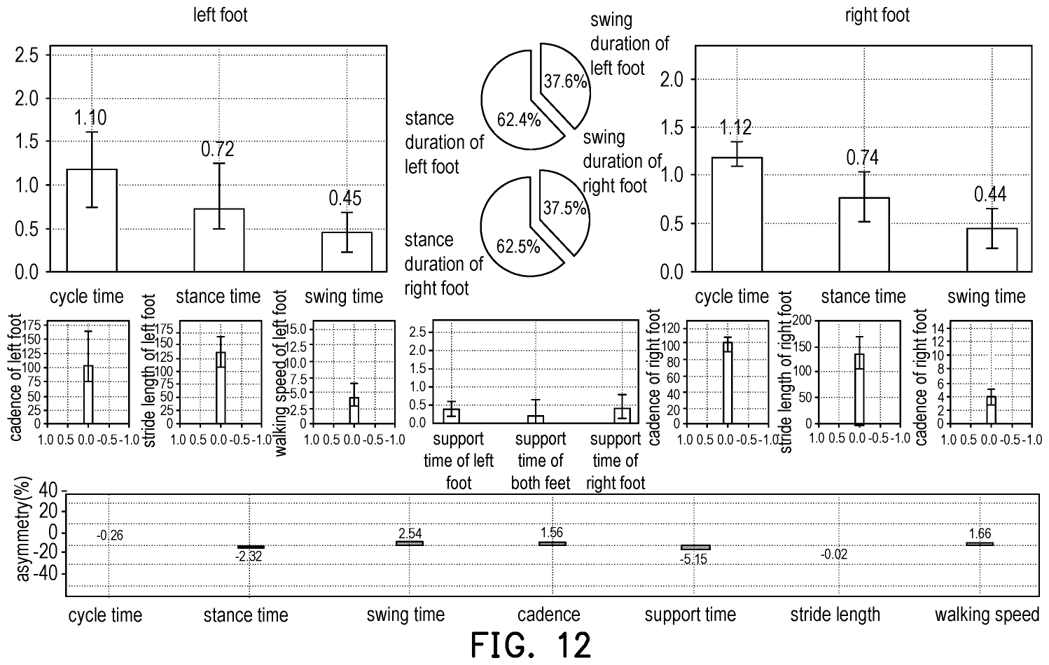

Please refer to FIG. 12. FIG. 12 is a schematic diagram of a gait index dashboard according to an embodiment of the present disclosure. In FIG. 12, the processor 104 may present each gait index of both feet in a corresponding pie chart or bar chart. In addition, the processor 104 may further present the gait asymmetry when the subject under test is walking with both feet in the following asymmetry indices. As mentioned above, the lower the asymmetry of the feet of the subject under test in a certain gait index, the better the symmetry of the feet of the subject under test in this gait index.

For example, since the asymmetry corresponding to cycle time and stride length in FIG. 12 is relatively low, it may be obtained that the cycle time and stride length of both feet of the subject under test have better symmetry when the subject under test is walking. Moreover, since the asymmetry corresponding to the support time in FIG. 12 is relatively high, which is −5.15, it can be obtained that the support time of the feet of the subject under test has a poor symmetry when the subject under test is walking, indicating that the user spends a little more time using the right foot for support than the left foot. In FIG. 12, the asymmetry corresponding to the swing duration is 2.54, indicating that the user swings the left foot in the air a little longer than the right foot.

In summary, the method provided in the embodiments of the present disclosure is able to find out the gait events and corresponding event time points belonging to the same step based on the related probability distributions after collecting motion data of the subject under test when the subject under test is walking. Based on the above, the method of the embodiments of the present disclosure may not only be adapted to the subjects under test who walk in different ways, but also may measure the gait events more accurately.

Although the present disclosure has been disclosed as above with embodiments, it is not intended to limit the present disclosure. Those with ordinary knowledge in the technical field may make some modifications and changes without departing from the spirit and scope of the present disclosure. Therefore, the scope to be protected by the present disclosure should be defined by the scope of the appended claims.

What is claimed is:

1. A gait analysis method, adaptable for a gait analysis device, and comprising:
obtaining consecutive N motion data, wherein N is a positive integer;
determining a plurality of probability distributions based on the N motion data, wherein the plurality of probability distributions respectively correspond to a plurality of gait events; and
determining an event time point of each of the gait events belonging to a specific step according to the plurality of probability distributions.

2. The method according to claim 1, wherein the N motion data is derived from a motion sensor set or worn on a foot, and each of the motion data comprises 6 degrees of freedom values.

3. The method according to claim 1, wherein determining the plurality of probability distributions based on the N motion data comprises:
feeding the N motion data into an artificial intelligence model, wherein the artificial intelligence model generates the plurality of probability distributions in response to the N motion data.

4. The method according to claim 3, wherein a training process of the artificial intelligence model comprises:
obtaining consecutive N reference motion data, and obtaining a plurality of data time points corresponding to each of the gait events from the N reference motion data;
generating a reference probability distribution corresponding to each of the gait events based on the plurality of data time points corresponding to each of the gait events;
training the artificial intelligence model based on the N reference motion data and the reference probability distribution, wherein the artificial intelligence model generates a prediction probability distribution corresponding to each of the gait events in response to the N reference motion data;
updating a plurality of model parameters of the artificial intelligence model based on a comparison result between the reference probability distribution and the prediction probability distribution corresponding to each of the gait events.

5. The method according to claim 4, wherein the plurality of gait events comprise a j-th gait event, and generating the reference probability distribution corresponding to each of the gait events based on the plurality of data time points corresponding to each of the gait events comprises:
finding out at least one specific data time point corresponding to the j-th gait event in the N reference motion data;
copying a preset probability distribution template to each of the specific data time points corresponding to the j-th gait event, generating the reference probability distribution corresponding to the j-th gait event.

6. The method according to claim 1, wherein the plurality of probability distributions comprise an i-th probability distribution, and determining the event time point of each of the gait events belonging to the specific step according to the plurality of probability distributions comprises:
determining a preset probability distribution template;
determining a plurality of probability distribution segments on the i-th probability distribution according to the preset probability distribution template, wherein i is an index value;
determining an intersection over union (IoU) between the preset probability distribution template and each of the probability distribution segments on the i-th probability distribution, and generating an IoU variation diagram corresponding to the i-th probability distribution accordingly;
determining the event time point of each of the gait events belonging to the specific step based on the IoU variation diagram corresponding to each of the probability distributions.

7. The method according to claim 6, wherein the preset probability distribution template is intercepted from a normal distribution with a specific standard deviation and a specific mean value.

8. The method according to claim 6, wherein determining the event time point of each of the gait events belonging to the specific step based on the IoU variation diagram corresponding to each of the probability distributions comprises:
combining the IoU variation diagram corresponding to each of the probability distributions into a reference IoU variation diagram, wherein the reference IoU variation diagram comprises a plurality of IoU sampling points;
finding out a plurality of sampling point groups from the reference IoU variation diagram, wherein the IoU corresponding to the plurality of IoU sampling points in each of the sampling point groups is higher than a preset threshold;
determining the gait event corresponding to each of the sampling point groups;
in response to determining that there are a plurality of specific sampling point groups satisfying a preset condition in the plurality of sampling point groups, determining the event time point of each of the gait events belonging to the specific step based on the plurality of specific sampling point groups.

9. The method according to claim 8, wherein the plurality of gait events comprise a 1-st gait event to a K-th gait event in sequence, and the method comprises:
in response to determining that consecutive K sampling point groups in the plurality of sampling point groups correspond to the 1-st gait event to the K-th gait event in sequence, determining that the consecutive K sampling point groups are the specific sampling point groups satisfying the preset condition.

10. The method according to claim 8, wherein the plurality of specific sampling point groups comprise a j-th specific sampling point group, and determining the event time point of each of the gait events based on the plurality of specific sampling point groups comprises:
determining that the plurality of specific sampling point groups belong to the specific step;
finding out a specific sampling point from the j-th specific sampling point group, wherein the j-th specific sampling point group corresponds to a j-th gait event in the plurality of gait events, and j is an index value;
taking a time point corresponding to the specific sampling point of the j-th specific sampling point group as the event time point of the j-th gait event belonging to the specific step.

11. The method according to claim 1, wherein the plurality of gait events comprise a 1-st gait event to a K-th gait event in sequence, and the method further comprises:
in response to determining that the event time point of each of the gait events corresponding to a specific step has been found based on the N motion data, obtaining the event time point of the K-th gait event corresponding to the specific step;
obtaining other consecutive N motion data from the event time point of the K-th gait event corresponding to the specific step, and determining a plurality of other probability distributions based on the N other motion data, wherein the plurality of other probability distributions respectively correspond to the plurality of gait events; and
determining the event time point of each of the gait events belonging to another specific step according to the plurality of other probability distributions.

12. The method according to claim 1, further comprising:
in response to determining that the event time point of each of the gait events corresponding to the specific step is not found based on the N motion data, obtaining other consecutive N motion data, wherein a first other motion data among the N other motion data has a time difference with a first motion data among the N motion data;
determining a plurality of other probability distributions based on the N other motion data, wherein the other probability distributions respectively correspond to the plurality of gait events; and
determining the event time point of each of the gait events belonging to another specific step according to the plurality of other probability distributions.

13. The method according to claim 1, wherein the specific step corresponds to a first foot of a subject under test, and after determining the event time point of each of the gait events belonging to the specific step according to the plurality of probability distributions, the method further comprises:
determining at least one first gait index corresponding to the first foot according to the event time point of each of the gait events belonging to the specific step;
obtaining at least one second gait index of a second foot corresponding to the subject under test;
determining a gait asymmetry between the first foot and the second foot based on the at least one first gait index and the at least one second gait index.

14. The method according to claim 1, wherein the plurality of gait events comprise an initial contact, a foot-flat, a heel-off and a toe-off.

15. A gait analysis device, comprising:
a storage circuit, which stores a program code;
a processor, which is coupled to the storage circuit to access the program code to:
obtain consecutive N motion data, wherein N is a positive integer;
determine a plurality of probability distributions based on the N motion data, wherein the plurality of probability distributions respectively correspond to a plurality of gait events; and
determine an event time point of each of the gait events belonging to a specific step according to the plurality of probability distributions.

16. The gait analysis device according to claim 15, wherein the N motion data is derived from a motion sensor set or worn on a foot, and each of the motion data comprises 6 degrees of freedom values.

17. The gait analysis device according to claim 15, wherein the plurality of probability distributions comprise an i-th probability distribution, and the processor is configured to:
determine a preset probability distribution template;
determine a plurality of probability distribution segments on the i-th probability distribution according to the preset probability distribution template, wherein i is an index value;
determine an intersection over union (IoU) between the preset probability distribution template and each of the probability distribution segments on the i-th probability distribution, and generating an IoU variation diagram corresponding to the i-th probability distribution accordingly; and
determine the event time point of each of the gait events belonging to the specific step based on the IoU variation diagram corresponding to each of the probability distributions.

18. The gait analysis device according to claim 15, further comprising a display device, wherein the specific step corresponds to a first foot of a subject under test, and after determining the event time point of each of the gait events belonging to the specific step according to the plurality of probability distributions, the processor is further configured to:
determine at least one first gait index corresponding to the first foot according to the event time point of each of the gait events belonging to the specific step;
obtain at least one second gait index of a second foot corresponding to the subject under test;
determine a gait asymmetry between the first foot and the second foot based on the at least one first gait index and the at least one second gait index;
control the display device to display the gait asymmetry.

19. A computer-readable storage medium, wherein the computer-readable storage medium records an executable computer program, and the executable computer program is loaded by a gait analysis device to perform the following:
obtaining consecutive N motion data, wherein N is a positive integer;
determining a plurality of probability distributions based on the N motion data, wherein the plurality of probability distributions respectively correspond to a plurality of gait events; and
determining an event time point of each of the gait events belonging to a specific step according to the plurality of probability distributions.

20. The computer-readable storage medium according to claim 19, wherein the specific step corresponds to a first foot of a subject under test, and after determining the event time point of each of the gait events belonging to the specific step according to the plurality of probability distributions, further comprising:
determining at least one first gait index corresponding to the first foot according to the event time point of each of the gait events belonging to the specific step;
obtaining at least one second gait index of a second foot corresponding to the subject under test;
determining a gait asymmetry between the first foot and the second foot based on the at least one first gait index and the at least one second gait index.
